(19) United States
(12) Patent Application Publication
(10) Pub. No.: US 2009/0162881 A1
(43) Pub. Date: Jun. 25, 2009

(54) METHOD OF MEASURING ADENINE NUCLEOTIDE

(75) Inventors: Daisuke Okamura, Ehime (JP); Koji Sode, Tokyo (JP); Wakako Tsugawa, Tokyo (JP)

Correspondence Address: PEARNE & GORDON LLP, 1801 EAST 9TH STREET, SUITE 1200, CLEVELAND, OH, 44114-3108, US

(73) Assignees: MATSUSHITA ELECTRIC INDUSTRIAL CO., LTD., Osaka (JP); TOKYO UNIVERSITY OF AGRICULTURE AND TECHNOLOGY, Tokyo (JP)

(21) Appl. No.: 12/093,521

(22) PCT Filed: Nov. 10, 2006
(86) PCT No.: PCT/JP2006/322490

§ 371 Date: Aug. 4, 2008

(30) Foreign Application Priority Data
Nov. 14, 2005 (JP) 2005-328962

(51) Int. Cl. C12Q 1/48; C12Q 1/26

(52) U.S. Cl. 435/15; 435/25; 435/26

(57) ABSTRACT

A high sensitivity electrochemistry type method for measuring adenine nucleotide which has a convenient and further miniaturized measuring device structure; is low in consumptive power; and does not require a treatment operation for substances that cause turbidity is provided. A method for measuring adenine nucleotide, which comprises a step A for converting adenosine triphosphate to adenosine diphosphate by an enzyme E₁, a step B for converting said adenosine diphosphate and a phosphate donor P₂ to adenosine triphosphate and dephosphorylated phosphate donor P_{2′} by an enzyme E₂, and a step C for electrochemically measuring said donor P_{2′} by carrying out an oxidation-reduction reaction.

### FIELD OF THE INVENTION

The present invention relates to a method for measuring so-called adenosine nucleotide such as adenosine triphosphate (to be referred to as ATP hereinafter), adenosine diphosphate (to be referred to as ADP hereinafter), adenosine monophosphate (to be referred to as AMP hereinafter) or a mixture thereof. More illustratively, it relates to a technique for electrochemically measuring adenosine nucleotide with good sensitivity.

### BACKGROUND OF THE INVENTION

Adenosine nucleotide relates to the energy metabolism in the living organism. Various chemical reactions in the living organism are carried out in many cases by using the energy released when ATP is hydrolyzed and converted into ADP or AMP. Additionally, adenine nucleotide is also used in the living organism as a precursor of ribonucleic acid (RNA), a phosphate donor of the phosphorylation reaction in the living organism and the like.

Thus, since adenine nucleotide is a compound which takes an extremely important role in the living organism, measurement of adenine nucleotide performs an important role in various fields.

For example, regarding ATP as one of the adenine nucleotide, ATP concentration is used in the field of food hygiene as an index of the degree of pollution with bacteria and the like microorganism, food residues as a hotbed of microbial pollution and the like. Additionally, it is used as various indexes such as monitoring of the number of viable cells and metabolic changes in cells, quality control of food including the degree of maturity, the degree of putrefaction and the like of food, water analysis for the water purification and the like.

A bioluminescence method which uses the luciferin-luciferase reaction has been known as a method for measuring ATP. The method effects luminescence by allowing luciferin and luciferase to react with ATP extracted from a sample, in the presence of a divalent metal ion. Since one photon per one molecule is released by the luminescence, ATP can be quantitatively detected by integrating the values based on the duration of luminescence.

However, while the luciferin-luciferase bioluminescence method has an advantage in that ATP can be measured quickly, it has a problem of poor radiation stability since the radiation disappears within very short time. Therefore, it is necessary to use a measuring device which can strictly control the reaction time and has an auto-injection function for capturing the luminescence which disappears within a short period of time in order to obtain sensitivity and accuracy.

Accordingly, a method for extending the luminescent decay time by temporarily generating a strong luminescence through the addition of pyrophosphoric acid to the reaction system at the time when the bioluminescence reaction proceeded to a certain degree and the luminescence quantity reduced and thereby again increasing the peak strength of light in the middle of the luminescence reaction (cf. Non-patent Reference 1) has been devised. However, because of the absence of new regeneration of ATP, the luminescence is periodically attenuated as the ATP is consumed so that the luminescent decay time cannot be stabilized over a prolonged period of time.

For the purpose of resolving the aforementioned problem regarding the luciferin-luciferase luminescence stability, a method for obtaining luminescence stability without attenuating the luminescence by forming an ATP regeneration reaction system (cf. Patent Reference 1) has been devised.

In the method, a reaction for forming ATP, pyruvic acid and phosphoric acid by allowing pyruvate orthophosphate di-kinase to react with AMP, pyrophosphoric acid, phosphoenolpyruvic acid and magnesium ion (Reaction 1) is carried out. Subsequently, a reaction for forming AMP, pyrophosphoric acid, oxyl-luciferin, carbon dioxide and light is carried out by allowing luciferase to react with ATP, luciferin, dissolved oxygen and magnesium ion (Reaction 2). By combining the aforementioned Reaction 1 and Reaction 2 and cycling the reactions, the ATP regeneration reaction system is formed. Thus, the luminescence stability can be obtained. The reaction is shown below.

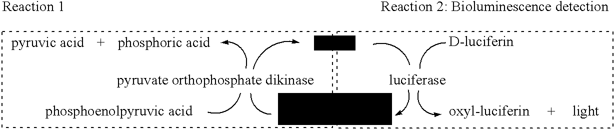

Additionally, a method for detecting an extremely small amount of ATP has also been devised (cf. Patent Reference 2), wherein a reaction in which AMP and myokinase are allowed to react with ATP in a sample to be converted into two molecules of ADP (Reaction 3) and a reaction in which ADP and polyphosphate kinase react in the presence of a polyphosphoric acid compound to effect conversion into ATP and polyphosphoric acid compounds (Reaction 4) are used; the Reaction 3 and Reaction 4 are regarded as a pair of reaction systems; ATP is amplified by the second power according to the frequency of the reactions; and the amplified ATP is detected by a bioluminescence method. The reaction is shown below.

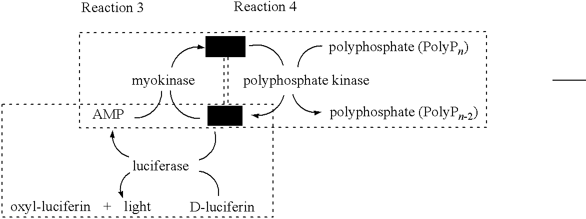

On the other hand, an ATP measuring method which uses glucose oxidase and hexokinase has been proposed as an electrochemical measuring method (cf. Patent Reference 3). The measuring method uses that the ratio of the competitive reaction of glucose oxidase and hexokinase for glucose depends on the amount of ATP.

Additionally, an ATP measuring method has also been proposed (cf. Patent Reference 4 and Patent Reference 5), in which ATP is hydrolyzed by using ATP hydrolase (ATPase) to form a hydrogen ion and its pH change is detected by a hydrogen ion-sensitive electric field effect type transistor.

Non-patent Reference 1: Arch. Biochem. Biophys. 46, 399-416; 1953

### Patent Reference 1: JP-A-9-234099

### Patent Reference 2: JP-A-2001-299390

### Patent Reference 3: JP-A-60-17347

### Patent Reference 4: JP-A-61-122560

### Patent Reference 5: JP-A-61-269058

### DISCLOSURE OF THE INVENTION

### Problems to be Solved by the Invention

However, the aforementioned prior art technique based on the bioluminescence method has problems that not only the luminescence detecting device is expensive but also optical system parts become necessary and, as a result, the device structure is complex.

Additionally, since ATP is detected by a luminescence detection method in the method, there is a problem in that consumptive power of the measuring device is large.

Next, since luminescence measurement is possible in the bioluminescence method only when dissolved oxygen is present in the sample, a sample in which dissolved oxygen is not present cannot be measured.

Furthermore, in general, it is difficult to apply an assay method by the luminescence detection method to samples having turbidity. Accordingly, it is difficult to measure samples having high turbidity such as milk and blood directly by the aforementioned method. Therefore, there is a problem that it is necessary to carry out a removing treatment or dissolving treatment of substances which is main cause of the turbidity or to dilute the samples prior to the measurement.

The aforementioned electrochemistry type measuring method as a prior art technique has advantages that the problems of the bioluminescence method cam be solved and, furthermore, samples having no dissolved oxygen can be measured by the use of an electron mediator, while its ATP measuring sensitivity is merely about 10⁻⁴ to 10⁻⁶ M which does not reach the sensitivity practically used in the field of food hygiene and the like. Therefore, an electrochemistry type ATP measuring method having good sensitivity has been in desired.

The object of the present invention is to solve the aforementioned problems of the prior art and provide a high sensitivity electrochemistry type measuring method of adenine nucleotide which has a convenient and further miniaturized measuring device structure; is low in consumptive power; and does not require a pretreatment operation for substances which cause turbidity which is necessary in the bioluminescence method.

### Means for Solving the Problems

The present inventors have found that the aforementioned problems can be resolved by the following constructions.

[1] A method for measuring adenine nucleotide, which comprises

a step A for converting adenosine triphosphate to adenosine diphosphate by an enzyme E₁;

a step B for converting said adenosine diphosphate and a phosphate donor P₂ to adenosine triphosphate and dephosphorylated phosphate donor P₂′ by an enzyme E₂; and

a step C for electrochemically measuring said donor P_{2′} by carrying out an oxidation-reduction reaction of said donor P_{2′}.

[2] The method for measuring adenine nucleotide according to [1], wherein the adenosine triphosphate and the dephosphorylated phosphate donor P_{2′} are formed according to the frequency of the reactions by repeating a cycle consisting of the step A and step B two or more times to carry out a reaction.

[3] The method for measuring adenine nucleotide according to [1] or [2], wherein the dephosphorylated phosphate donor P_{2′} formed by step B is measured as the amount of adenine nucleotide.

[4] The method for measuring adenine nucleotide according to any one of [1] to [3], wherein myokinase is used as the enzyme E₁.

[5] The method for measuring adenine nucleotide according to any one of [1] to [4], wherein the oxidation-reduction reaction in the step C is carried out by an oxidation-reduction enzyme E₃.

[6] The method for measuring adenine nucleotide according to any one of [1] to [5], wherein the donor P_{2′} is electrochemically measured in the step C by carrying out an oxidation-reduction reaction of said donor P_{2′} which consumes oxygen molecule and detecting amount of the consumed oxygen.

[7] The method for measuring adenine nucleotide according to any one of [1] to [5], wherein the donor P_{2′} is electrochemically measured in the step C by carrying out an oxidation-reduction reaction of said donor P_{2′} which produces hydrogen peroxide and detecting amount of the produced hydrogen peroxide.

[8] The method for measuring adenine nucleotide according to any one of [1] to [5], wherein the electrochemically detecting way in the step C uses an electron mediator as the electron acceptor.

[9] The method for measuring adenine nucleotide according to [1], wherein pyruvate kinase is used as the enzyme E₂, and pyruvate oxidase is used as the enzyme E₃.

[10] The method for measuring adenine nucleotide according to [1], wherein pyruvate kinase is used as the enzyme E₂, and pyruvate dehydrogenase is used as the enzyme E₃.

[11] The method for measuring adenine nucleotide according to [1], wherein hexokinase or glucokinase is used as the enzyme E₂, and glucose oxidase is used as the enzyme E₃.

### EFFECT OF THE INVENTION

According to the method for measuring adenine nucleotide of the present invention, the measuring device structure becomes simple and convenient so that an electrochemistry type measuring device which is further miniaturized and has low consumptive power can be provided, and even a turbid sample can be easily measured.

Additionally, since amounts of ATP and a dephosphorylated phosphate donor are amplified by repeating two or more times of a reaction system which is a pair of the aforementioned step A and step B, and the dephosphorylated phosphate donor is detected as the amount of adenine nucleotide, the measurement can be carried out with good sensitivity.

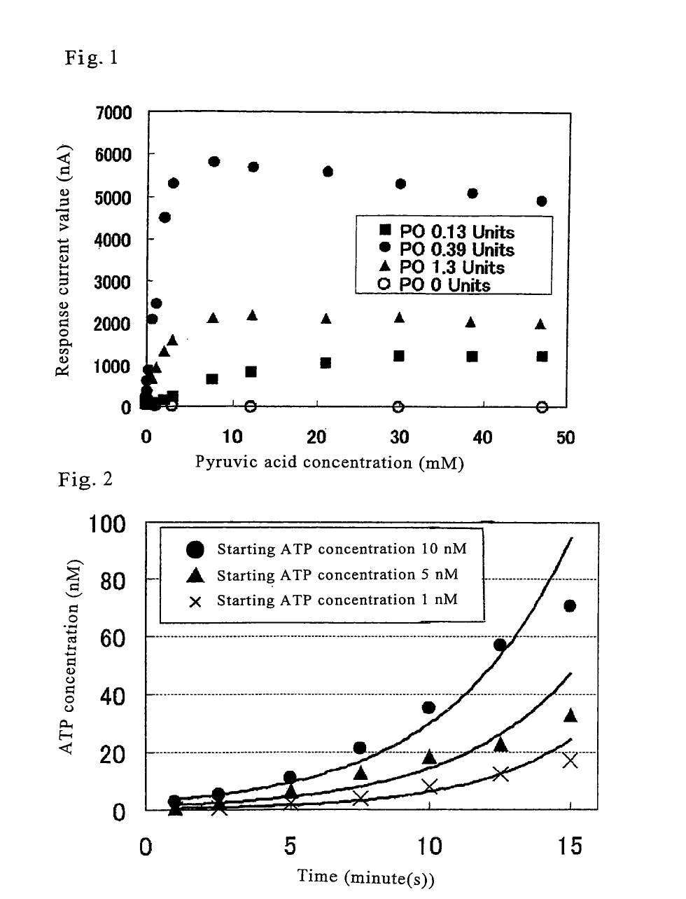

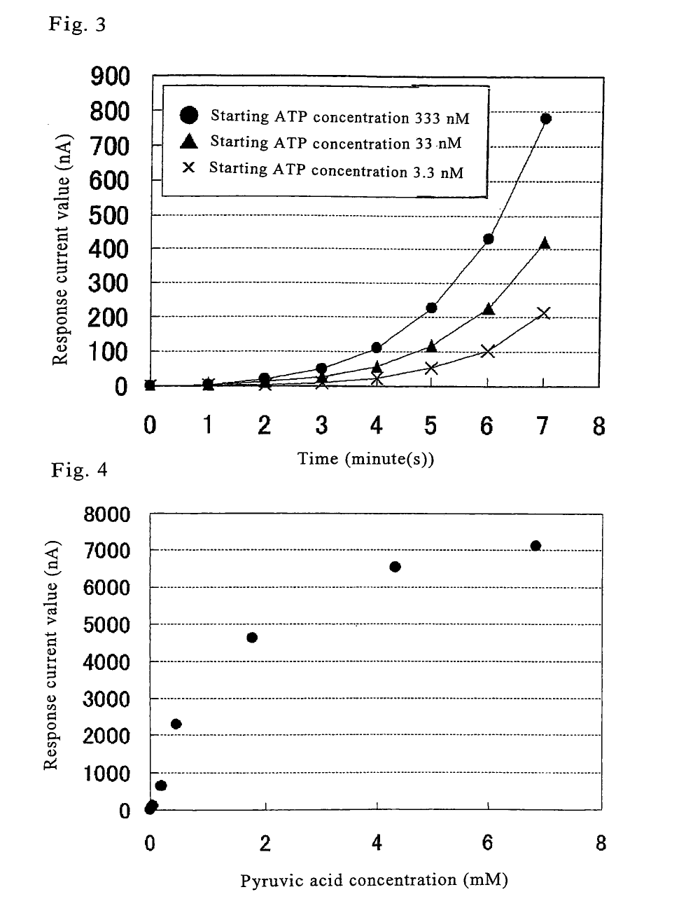

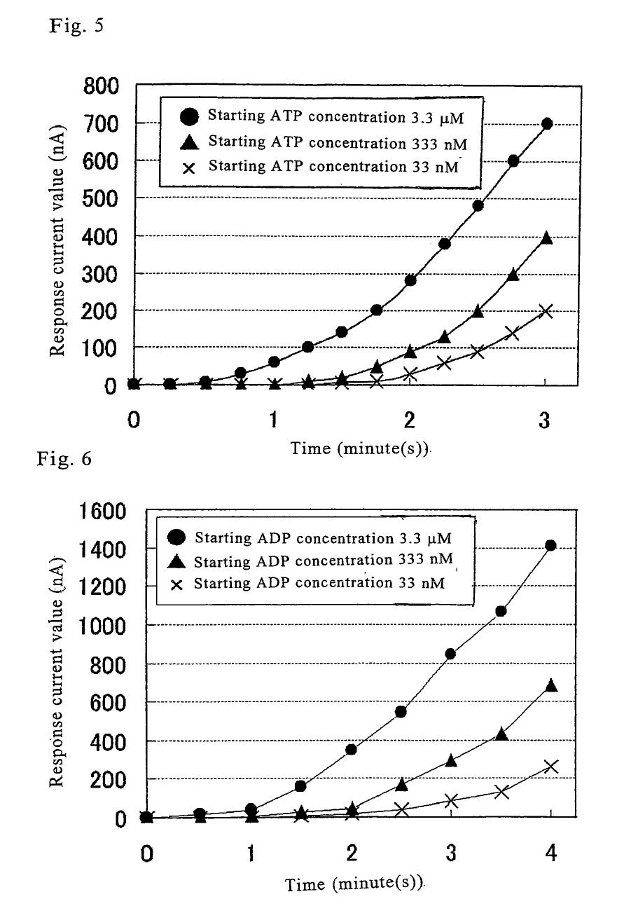

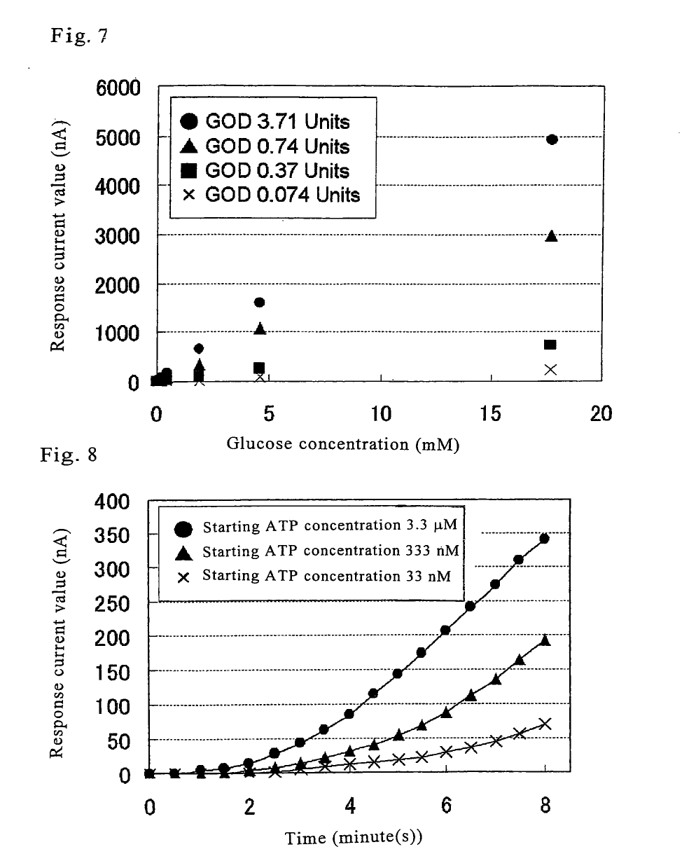

### BRIEF DESCRIPTION OF THE DRAWINGS

[FIG. 1] A graph which shows a result of the evaluation of the pyruvate oxidase electrode in Example 1 of the present invention.

[FIG. 2] A graph which shows a result of the evaluation of the amplification of ATP in Example 1 of the present invention.

[FIG. 3] A graph which shows a result of the electrochemical measurement of ATP using the pyruvate oxidase electrode in Example 1 of the present invention.

[FIG. 4] A graph which shows a result of the evaluation of the pyruvate dehydrogenase electrode in Example 2 of the present invention.

[FIG. 5] A graph which shows a result of the electrochemical measurement of ATP using the pyruvate dehydrogenase electrode in Example 2 of the present invention.

[FIG. 6] A graph which shows a result of the electrochemical measurement of ADP using the pyruvate dehydrogenase electrode in Example 2 of the present invention.

[FIG. 7] A graph which shows a result of the evaluation of the glucose oxidase electrode in Example 3 of the present invention.

[FIG. 8] A graph which shows a result of the electrochemical measurement of ATP using the glucose oxidase electrode in Example 3 of the present invention.

### BEST MODE FOR CARRYING OUT THE INVENTION

The following illustratively describes embodiments of the method for measuring adenine nucleotide of the present invention.

The following is a scheme showing an outline of the measuring method of the present invention.

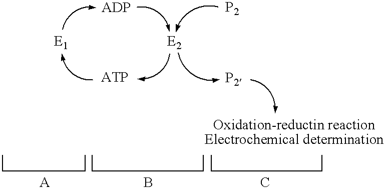

The method or device for measuring adenine nucleotide of the present invention comprises the following steps A to C or ways for carrying out the same:

step A for converting ATP to ADP by an enzyme E₁;

step B for converting ADP and a phosphate donor P₂ to ATP and dephosphorylated phosphate donor P_{2′} by an enzyme E₂; and

step C for electrochemically measuring the donor P_{2′} by carrying out an oxidation-reduction reaction of the donor P_{2′}.

In this connection, the adenine nucleotide according to the present invention means each of mainly adenosine triphosphate (ATP), adenosine diphosphate (ADP), adenosine monophosphate (AMP) and analogs thereof, or a combination thereof.

Additionally, the measurement according to the present invention means for example a detection for confirming the presence or absence, a determination for measuring the existing amount or the like.

ATP is converted to ADP in the step A by an enzyme E₁, and ADP and a phosphate donor P₂ are converted to ATP and a dephosphorylated phosphate donor P_{2′} in the step B by an enzyme E₂. Subsequently, when the step A and step B become a pair and reaction of the pair occurs n times, n molecules of the dephosphorylated phosphate donor P_{2′} are formed. In this manner, by allowing the phosphate donor P₂ to be present excessively, the dephosphorylated phosphate donor P_{2′} is formed in proportion to the amount of the adenine nucleotide presenting in the reaction system (total amount of ATP and ADP in this case), and amount of the adenine nucleotide in the inspecting object can be calculated by measuring amount of the dephosphorylated phosphate donor P_{2′} by an oxidation-reduction reaction.

Also, since the step A and step B repeatedly generate the reaction as a pair as described in the above, amount of the dephosphorylated phosphate donor P_{2′} increases also in proportion to the period of time. By this, even in a case of a sample of extremely low concentration, the sample can be measured at a high sensitivity.

Also, it is preferable that the step A is a step in which AMP and ATP are converted to two molecules of ADP by an enzyme E₁. In the case, since the enzyme reactions of step A and step B become a pair and 2ⁿ of the donor P_{2′} are formed by carrying out n times of the enzyme reactions of the pair, the sensitivity can be largely improved within a short period of time. In the case, total amount of ATP and ADP in the analysis subject can be measured by allowing AMP to exist in an excess amount in addition to the phosphate donor P₂.

Additionally, according to the present invention, since it is possible to carry out an electrochemical measurement by mediating the step C, the measurement can be carried out within a short period of time using a simple device.

The following illustratively describes each step.

Firstly, the step A is described in detail.

The enzyme E₁ to be used in the step A is not particularly limited with the as long as it is an enzyme which converts ATP into ADP as described in the above, and the followings can be cited as examples.

1) An enzyme belonging to EC (Enzyme Code) 2.7., which transfers a phosphorus-containing group and converts ATP into ADP.

2) An enzyme belonging to EC 3.5., which acts on a C—N bond other than peptide bond and converts ATP into ADP.

3) An enzyme belonging to EC 3.6., which acts on an acid anhydride and converts ATP into ADP.

4) An enzyme belonging to EC 4.1., which acts on a carbon-carbon bond and converts ATP into ADP.

5) An enzyme belonging to EC 4.6., which acts on a phosphorus-carbon bond and converts ATP into ADP.

6) An enzyme belonging to EC 6.2., which forms a carbon-sulfur bond and converts ATP into ADP.

7) An enzyme belonging to EC 6.3., which forms a carbon-nitrogen bond and converts ATP into ADP.

8) An enzyme belonging to EC 6.4., which synthesizes a phosphate ester bond and converts ATP into ADP.

Additionally, an enzyme which produces 2 ADP using AMP and ATP as the substrates as described in the above is also desirable. As such an enzyme, myokinase can be cited as an example.

Next, the step B is described in detail.

In the step B, it is necessary that the phosphate donor P₂ is allowed to exist excessively based on the reaction system. In this case, the excess amount means an amount sufficient for subjecting to the electrochemical measuring method which is described later, and can be optionally set according to the measuring object and analyte.

The phosphate donor P₂ is not particularly limited, as long as it becomes the substrate of the enzyme E₂ together with ADP to form ATP and a dephosphorylated phosphate donor P_{2′}, and can be optionally selected together with the enzyme E₂.

Preferable combination of the phosphate donor P₂ and enzyme E₂ is described later.

Next, the step C is described in detail.

The step C is a step in which the donor P_{2′} is electrochemically measured by subjecting the dephosphorylated phosphate donor P_{2′} to an oxidation-reduction reaction.

The oxidation-reduction reaction is carried out by allowing an oxidized electron acceptor P₃ to react with the dephosphorylated phosphate donor P_{2′} to effect oxidation of the dephosphorylated phosphate donor P_{2′} and resulting conversion of the electron acceptor into reduced P_{3′}. According to the present invention, since amount of the reduced electron acceptor is also proportional to the amount of adenine nucleotide (basically becomes the same), it becomes possible to know the amount of adenine nucleotide by measuring amount of the reduced electron acceptor by an electrochemical measuring means. It is preferable that the reduced electron acceptor is again converted into the oxidized electron acceptor by the electrochemical measuring ways and used circulatory in the reaction of step C. In this connection, although it is necessary that the oxidized electron acceptor is also present excessively in the reaction system similar to the case of the phosphate donor P₂, when the electron acceptor is recycled by the electrochemical measuring ways, its amount can be set by taking it into consideration.

An enzyme reaction which is catalyzed by an enzyme E₃ is preferable as the oxidation-reduction reaction in the case. The enzyme E₃ is optionally selected according to the kinds of the donor P_{2′} to be used as the substrate.

In the step C, it is preferable to use an electron mediator as the electron acceptor.

The electron mediator is a compound which catalyzes electron transfer between the enzyme and electrode in the oxidation-reduction enzyme reaction, and is preferable since it enables to measure a sample in which dissolved oxygen does not exist.

Specifically, for example, it is preferable to use potassium ferricyanide/potassium ferrocyanide, 1-methoxy-5-methylphenaziniummethyl sulfate (mPMS) and the like.

Additionally, it is also preferable to subject said donor P_{2′} to an oxidation-reduction reaction which consume oxygen molecule and detect amount of the consumed oxygen, since the known way to measure oxygen molecule can be used.

Alternatively, it is also preferable to subject said donor P_{2′} to an oxidation-reduction reaction which produces hydrogen peroxide and measure the produced hydrogen peroxide electrochemically.

In this case, examples of electrochemical way for measuring include a way which uses current, voltage, quantity of electricity, impedance and the like.

By the electrochemical measurement, it becomes possible to carry out the measurement using a device which is simple in comparison with the conventional measuring means such as bioluminescence and the like as described in the foregoing. Additionally, it also becomes possible to easily determine a substrate contained in an optional measuring object, for example by a method in which a calibration curve is prepared in advance by measuring the electrochemical quantity detected by the means for measuring of the present invention for each amount of the substrate.

Although preferable combinations of the phosphate donor P₂ and enzyme E₂ and enzyme E₃ and other substrates are shown in the following, the present invention is not limited thereto.

### (1) E₂: Pyruvate Kinase

(ADP+phosphoenolpyruvic acid→ATP+pyruvic acid)

E₃: pyruvate oxidase

(pyruvic acid+O₂+phosphoric acid+H₂O→acetyl phosphate+H₂O₂+CO₂)

### (2) E₂: Pyruvate Kinase

(ADP+phosphoenolpyruvic acid→ATP+pyruvic acid)

E₃: pyruvate dehydrogenase

(pyruvic acid+oxidized electron acceptor+phosphoric acid→acetyl phosphate+reduced electron acceptor+CO₂)

### (3) E₂: Glucokinase

(ADP+D-glucose 6-phosphate→ATP+D-glucose)

E₃: glucose oxidase

(D-glucose+O₂→D-glucono-1,5-lactone+H₂O₂)

### (4) E₂: Glucokinase

(ADP+D-Glucose 6-Phosphate→ATP+D-Glucose)

E₃: pyranose oxidase

(D-glucose+O₂→2-dehydro-D-glucose+H₂O₂)

### (5) E₂: Glucokinase

(ADP+D-Glucose 6-Phosphate→ATP+D-Glucose)

E₃: glucose dehydrogenase

(D-glucose+oxidized electron acceptor→D-glucono-1,5-lactone+reduced electron acceptor)

### (6) E₂: Galactokinase

(ADP+α-D-galactose 1-phosphate→ATP+D-galactose)

E₃: galactose oxidase

(D-galactose+O₂→D-galacto-hexodialdose+H₂O₂)

### (7) E₂: Gluconokinase

(ADP+6-Phospho-D-Gluconate→ATP+D-Gluconate)

E₃: gluconate-2-dehydrogenase

(D-gluconate+oxidized electron acceptor→2-dehydro-D-gluconate+reduced electron acceptor)

### (8) E₂: Dehydrogluconokinase

(ADP+6-phospho-2-dehydro-D-gluconate→ATP+2-dehydro-D-gluconate)

E₃: gluconate-2-dehydrogenase

(2-dehydro-D-gluconate+reduced electron acceptor→D-gluconate+oxidized electron acceptor)

### (9) E₂: Dehydrogluconokinase

(ADP+6-phospho-2-dehydro-D-gluconate→ATP+2-dehydro-D-gluconate)

E₃: dehydrogluconate dehydrogenase

(2-dehydro-D-gluconate+oxidized electron acceptor→2,5-didehydro-D-gluconate+reduced electron acceptor)

### (10) E₂: Adenosine Kinase

(ADP+AMP→ATP+Adenosine)

E₃: nucleoside oxidase

(adenosine+2 O₂→9-ribulonosyladenine+2H₂O₂)

### (11) E₂: Glycerone Kinase

(ADP+glycerone phosphate→ATP+glycerone)

E₃: glycerol dehydrogenase

(glycerone+reduced electron acceptor→glucerol+oxidized electron acceptor)

### (12) E₂: Glycerol Kinase

(ADP+sn-glycerol 3-phosphate→ATP+glycerol)

E₃: glycerol dehydrogenase

(glycerol+oxidized electron acceptor→glycerone+reduced electron acceptor)

### (13) E₂: Choline Kinase

(ADP+O-phosphocholine→ATP+choline)

E₃: choline oxidase

(choline+O₂→betaine aldehyde+H₂O₂)

### (14) E₂: Choline Kinase

(ADP+O-phosphocholine→ATP+choline)

E₃: choline dehydrogenase

(choline+oxidized electron acceptor→betaine aldehyde+reduced electron acceptor)

### (15) E₂: N-acetylglucosamine Kinase

(ADP+N-acetyl-D-glucosamine 6-phosphate→ATP+N-acetyl-D-glucosamine)

E₃: N-acylhexosamine oxidase

(N-acetyl-D-glucosamine+O₂→N-acetyl-D-glucosaminate+H₂O₂)

### (16) E₂: Ethanolamine Kinase

(ADP+O-phosphoethanolamine→ATP+ethanolamine)

E₃: ethanolamine oxidase

(ethanolamine+H₂O+O₂→glycoaldehyde+NH₃+H₂O₂)

### (17) E₂: β-Glucoside Kinase

(ADP+6-phospho-β-glucosyl-(1,4)-D-glucose→ATP+cellobiose)

E₃: cellobiose oxidase

(cellobiose+O₂→cellobiono-1,5-lactone+H₂O₂)

### (18) E₂: β-Glucoside Kinase

(ADP+6-phospho-β-glucosyl-(1,4)-D-glucose→ATP+cellobiose)

E₃: cellobiose dehydrogenase

(cellobiose+oxidized electron acceptor→cellobiono-1,5-lactone+reduced electron acceptor)

### (19) E₂: Thiamine Kinase

(ADP+thiamine phosphate→ATP+thiamine)

E₃: thiamine oxidase

(thiamine+2 O₂→thiamine acetate+2H₂O₂)

### (20) E₂: Xylitol Kinase

(ADP+xylitol 5-phosphate→ATP+xylitol)

E₃: xylitol oxidase

(xylitol+O₂→xylose+H₂O₂)

### (21) E₂: Aspartate Kinase

(ADP+4-phospho-L-aspartic acid→ATP+L-aspartic acid)

E₃: L-aspartate oxidase

(L-aspartic acid+H₂O+O₂→oxaloacetic acid+NH₃+H₂O₂)

### (22) E₂: Glutamate 5-Kinase

(ADP+L-glutamic acid 5-phosphate→ATP+L-glutamic acid)

E₃: L-glutamate oxidase

(L-glutamic acid+O₂+H₂O→2-Oxoglutarate+NH₃+H₂O₂)

### (23) E₂: Glutamate 1-Kinase

(ADP+α-L-glutamyl phosphate→ATP+L-glutamic acid)

E₃: L-glutamate oxidase

(L-glutamic acid+O₂+H₂O→oxoglutarate+NH₃+H₂O₂)

### (24) E₂: Ketohexokinase

(ADP+D-fructose 1-phosphate→ATP+D-fructose)

E₃: mannitol dehydrogenase

(D-fructose+oxidized electron acceptor→D-mannitol+reduced electron acceptor)

### (25) E₂: Fructokinase

(ADP+D-fructose 6-phosphate→ATP+D-fructose)

E₃: mannitol dehydrogenase

(D-fructose+oxidized electron acceptor→D-mannitol+reduced electron acceptor)

### (26) E₂: Choline Kinase

(ADP+O-phosphocholine→ATP+choline)

E₃: choline monooxygenase

(choline+O₂+2 reduced electron acceptors+2H⁺→betaine aldehyde hydrate+H₂+2 oxidized electron acceptors)

### (27) E₂: Glucuronokinase

(ADP+1-phospho-α-D-glucuronate→ATP+D-glucuronate)

E₃: inositol oxygenase

(D-glucuronate+H₂O→Myo-Inositol+O₂)

### (28) E₂: Uridine Kinase

(ADP+UMP→ATP+uridine)

E₃: pyrimidine-deoxynucleoside 2′-dioxygenase

(uridine+succinate+CO₂→2′-deoxyuridine+2-oxoglutarate+O₂)

### (29) E₂: Inositol-3-Kinase

(ADP+1D-myo-inositol 3-phosphate→ATP+myo-inositol)

E₃: inositol oxygenase

(myo-inositol+O₂→D-glucuronate+H₂O)

### (30) E₂: Inosine Kinase

(ADP+IMP→ATP+inosine)

E₃: nucleoside oxidase

(inosine+O₂→9-ribulonosylhypoxanthine+H₂O)

### (31) E₂: Acetate Kinase

(ADP+acetyl phosphate→ATP+acetic acid)

E₃: acetyl acetone dioxygenase

(acetic acid+2-oxopropanol→pentane-2,4 dione+−O₂)

Regarding the enzymes E₁ to E₃, those which are put on the market can be respectively used, or the enzymes purified or synthesized from the living organism by general methods can also be used. In using each enzyme, a stabilizer, a pH adjusting agent, a buffer and the like can be appropriately prepared according to the conventionally known optimum environment of the enzyme to be used.

Using amounts of the enzymes E₁ to E₃ are not particularly limited.

In this connection, according to the present invention, it is preferable for the sake of convenience to carry out the steps A to C in the same reaction system without interposing separation, purification and the like.

Additionally, each enzyme of the present invention may be respectively dissolved in the reaction system or immobilized. With regard to the immobilization, it can be carried out also by the general method described, for example, in “Seibutsu Kagaku Jikken Hou 28 Bioreactor Jikken Nyumon” (Gakkai Shuppan Center) and the like. Additionally, substrates such as phosphate donor P₂ may also be immobilized.

Examples of the immobilized reaction system include a system in which respective substrates and enzymes are immobilized on a thin film, a layered product thereof and the like.

Although the following describes the present invention further illustratively based on examples, the present invention is not limited thereto.

### EXAMPLE 1

### Enzyme Reaction Scheme 1

Step A: ATP+AMP→2 ADP (enzyme E₁: myokinase)
Step B: ADP+phosphoenolpyruvic acid→pyruvic acid+ATP (enzyme E₂: pyruvate kinase)
Step C: pyruvic acid+O₂+phosphoric acid+H₂O→acetyl phosphate+H₂O₂+CO₂ (enzyme E₃: pyruvate oxidase)

The enzyme reaction scheme 1 described in the above shows a case in which measurement of ATP as one of the adenine nucleotides was carried out in Example 1 of the present invention.

Firstly, ATP and AMP are converted into two molecules of ADP by the enzyme reaction of myokinase (Step A).

Next, two molecules of ATP and two molecules of pyruvic acid as the dephosphorylated phosphate donor are formed by allowing the thus formed two molecules of ADP and two molecules of phosphoenolpyruvic acid as the phosphate donor to react with pyruvate kinase (Step B).

By regarding the Step A and Step B as a pair of reaction systems and repeatedly carrying out the reactions two or more times, pyruvic acid is formed according to the second power of ATP and formation of ATP.

By regarding the pyruvic acid formed in response to the amplification of ATP as the amount of ATP and allowing pyruvic acid to react with pyruvate oxidase in the presence of oxygen, phosphoric acid and H₂O, formation of acetyl phosphate, hydrogen peroxide and carbon dioxide is carried out. By electrochemically detecting the hydrogen peroxide formed by the reaction with pyruvate oxidase, ATP is detected (Step C).

### (Preparation and Evaluation of Oxygen Electrode)

To 100 μl of stilbazolium-modified vinyl alcohol (PVA-SbQ) manufactured by Toyo Gosei Kogyo adjusted to 0.1 g/ml, pyruvate oxidase solution which is equivalent to 0.013 unit, 0.039 unit or 0.13 unit and derived from Aerococcus viridance and manufactured by MP Biomedicals are mixed, and 11.5 μl of the mixed liquid was added dropwise to the surface of a platinum (Pt) electrode and allowed to stand overnight at 4° C. to effect air-drying. After the air-drying, light of a fluorescent lamp was applied to the electrode for 15 minutes to effect optical bridging to prepare a pyruvate oxidase electrode.

Response current value at the time of the addition of pyruvic acid of each concentration was measured by applying +600 mV of impressed electric potential to the reaction solution 1 described in the following, by using the pyruvate oxidase electrode, a silver (Ag)/silver chloride (AgCl) as the reference electrode and a Pt wire as the counter electrode. In this connection, the measurement was carried out under conditions of 37° C. and pH 7.0. Additionally, similar response current value of a Pt electrode to which pyruvate oxidase was not immobilized was also measured as the control.

### <Reaction Solution 1>

(i) Magnesium chloride hexahydrate manufactured by Kanto Chemical Co., Inc.: 10 mM (final concentration)
(ii) Flavin adenine dinucleotide disodium N hydrate manufactured by Wako Pure Chemical Industries, Ltd.: 0.01 mM (final concentration)
(iii) Thiamin pyrophosphate chloride manufactured by MP Biomedicals Inc.: 0.2 mM (final concentration)
(iv) Phosphate buffer pH 7.0: 50 mM (final concentration)

The results of carrying out the evaluation are shown in FIG. 1. The PO in FIG. 1 indicates pyruvate oxidase. While the response current value was not observed by the Pt electrode to which pyruvate oxidase was not immobilized, the electrodes to which pyruvate oxidase was immobilized respectively showed increase in the current value according to the pyruvic acid concentrations. In this connection, the Pt electrode to which 0.13 unit or 0.39 unit of pyruvate oxidase was immobilized showed a pyruvic acid detection limit of about 24 μM. On the other hand, regarding the Pt electrode to which 1.3 unit of pyruvate oxidase was immobilized, the pyruvic acid detection limit was about 60 μM.

### (Verification of Amplification Reaction)

It was confirmed that ATP is amplified when the Step A (myokinase) and Step B (pyruvate kinase) of the present invention are regarded as a pair of reaction systems and the reactions are repeatedly carried out two or more times.

Firstly, 2 ml of the following reaction solution was prepared and the enzyme reaction was started. After commencement of the reaction, 90 μl of respective sample was collected at an interval of 1 minute and allowed to react with 90 μl of a luciferin-luciferase luminescence reagent manufactured by KIKKOMAN Corporation, and the ATP concentration was measured by a luminometer manufactured by TERUMO Corporation.

In this connection, the measuring conditions are pH 7.0 and room temperature (about 25° C.).

<Reaction solution 2>
(i) Myokinase derived from yeast and manufactured by Oriental Yeast Co., Ltd.: 0.047 U/ml (final concentration)
(ii) Pyruvate kinase derived from rabbit muscle and manufactured by MP Biomedicals Inc.: 5.6 U/ml (final concentration)
(iii) Phosphoenolpyruvic acid manufactured by Wako Pure Chemical Industries, Ltd.: 2 mM (final concentration)
(iv) Magnesium chloride hexahydrate manufactured by Kanto Chemical Co., Inc.: 10 mM (final concentration)
(v) Adenosine 1-phosphate manufactured by Wako Pure Chemical Industries, Ltd.: 1 mM (final concentration)
(vi) Adenosine 3-phosphate manufactured by Wako Pure Chemical Industries, Ltd: 1 nM, 5 nM or 10 nM (final concentration)
(vii) Phosphate buffer pH 7.0: 50 mM (final concentration)

A result in which respective sample showed different amplification according to the ATP concentrations was obtained (FIG. 2). Accordingly, it was shown that ATP and pyruvic acid are amplified when the enzyme reaction by myokinase as the Step A and the enzyme reaction by pyruvate kinase of Step B are regarded as a pair of reaction systems and the reactions are repeatedly carried out two or more times.

### (Measurement of ATP)

A pyruvate oxidase electrode was prepared by immobilizing 0.015 unit of pyruvate oxidase derived from Aerococcus viridance and manufactured by TOYOBO Co., Ltd. using the PVA-SbQ by the same aforementioned procedure.

Response current value was measured at an interval of 1 minute by preparing reaction solution 3; adding it to the 0.015 unit immobilized pyruvate oxidase electrode; and carrying out the reaction for 7 minutes by applying +600 mV of impressed electric potential thereto using a silver (Ag)/silver chloride (AgCl) as the reference electrode and a Pt wire as the counter electrode. In this connection, the measurement was carried out under conditions of 25° C. and pH 7.0, and the measured ATP concentrations were 333 nM, 33 nM and 3 nM.

<Reaction solution 3>
(i) Magnesium chloride hexahydrate manufactured by Kanto Chemical Co., Inc.: 10 mM (final concentration)
(ii) Flavin adenine dinucleotide disodium salt manufactured by MP Biomedicals Inc.: 0.01 mM (final concentration)
(iii) Thiamin pyrophosphate chloride manufactured by MP Biomedicals Inc.: 0.2 mM (final concentration)
(iv) Adenosine 1-phosphate manufactured by Wako Pure Chemical Industries Ltd.: 0.33 mM (final concentration)
(v) Adenosine 3-phosphate manufactured by Wako Pure Chemical Industries Ltd.: 333 nM, 33 nM, 3.3 nM (final concentration)
(vi) Phosphoenolpyruvic acid manufactured by Wako Pure Chemical Industries Ltd.: 0.33 mM (final concentration)
(vii) Myokinase derived from chick muscle and manufactured by SIGMA ALDRICH Corporation: 0.58 U/ml (final concentration)
(viii) Pyruvate kinase derived from rabbit muscle and manufactured by MP Biomedicals Inc.: 1.73 U/ml (final concentration)
(ix) Phosphate buffer pH 7.0; 50 mM (final concentration)

It can be seen that response current is found in each sample from 2 minutes after commencement of the reaction and amplified by different curves according to the respective ATP concentrations (FIG. 3). Based on the above, it can be seen that ATP is amplified when the aforementioned Step A and Step B are regarded as a pair of reaction systems and the reactions are repeatedly carried out two or more times. A very small amount of ATP can be electrochemically measured when the pyruvic acid formed according to the amplified ATP is subjected to an oxidation-reduction reaction by the Step C and the thus formed hydrogen peroxidase is electrochemically detected. Additionally, since samples having different ATP concentrations of 333 nM, 33 nM and 3.3 nM show respectively different amplifications, it is shown that ATP can be measured quantitatively.

### EXAMPLE 2

### Enzyme Reaction Scheme 2

Step A: ATP+AMP→2 ADP (enzyme E₁: myokinase)
Step B: ADP+phosphoenolpyruvic acid→pyruvic acid+ATP (enzyme E₂: pyruvate kinase)
Step C: pyruvic acid+oxidized electron acceptor+phosphoric acid→acetyl phosphate+reduced electron acceptor+CO₂ (enzyme E₃: pyruvate dehydrogenase)

The enzyme reaction scheme 2 described in the above shows a case in which measurement of the adenine nucleotide was carried out in Example 2 of the present invention.

In Example 1, pyruvate oxidase is used as the enzyme E₃ of the Step C, while pyruvate dehydrogenase is used in Example 2. Additionally, as the electron acceptor to be used in the oxidation-reduction reaction of Step C, 1-methoxy-5-methylphenaziniummethyl sulfate (mPMS) was used.

### (Preparation and Evaluation of Oxygen Electrode)

To the surface of a platinum (Pt) electrode having a diameter of 3 mm, 0.084 unit-equivalent pyruvate dehydrogenase derived from Lactobacillus and manufactured by TOYOBO Co., Ltd. was added dropwise and air-dried overnight at 4° C. After the air-drying, pyruvate dehydrogenase was immobilized to the Pt electrode surface by exposing the electrode surface to the steam of 25% glutaraldehyde solution manufactured by Wako Pure Chemical Industries Ltd. for about 30 minutes.

After the immobilization, the pyruvate dehydrogenase electrode was soaked in 10 mM Tris-HCl buffer of pH 7.0 to equilibrate the electrode surface.

Finally, it was soaked in 50 mM phosphate buffer of pH 7.0 to use it as the enzyme electrode in the measurement.

Response current values at the time of the addition of pyruvic acid having respective concentration was measured based on the following reaction solution 4, by applying +600 mV of impressed electric potential to the pyruvate dehydrogenase electrode, and to a silver (Ag)/silver chloride (AgCl) as the reference electrode and a Pt wire as the counter electrode. In this connection, the measurement was carried out under conditions of 25° C. and pH 7.0.

### <Reaction Solution 4>

(i) Magnesium chloride hexahydrate manufactured by Kanto Chemical Co., Inc.: 10 mM (final concentration)
(ii) Flavin adenine dinucleotide disodium salt manufactured by MP Biomedicals Inc.: 0.01 mM (final concentration)
(iii) Thiamin pyrophosphate chloride manufactured by MP Biomedicals Inc.: 0.2 mM (final concentration)
(iv) 1-Methoxy-5-methylphenaziniummethyl sulfate manufactured by Dojindo Laboratories: 10 mM (final concentration)
(v) Phosphate buffer pH 7.0; 50 mM (final concentration)

The results of carrying out the evaluation are shown in FIG. 4.

As a result of the evaluation of the pyruvate dehydrogenase electrode, increase in the current value according to the pyruvic acid concentrations was found. In this connection, the pyruvic acid detection limit was about 20 μM.

### (Measurement of ATP)

The pyruvate dehydrogenase electrode wherein 0.084 unit of pyruvate dehydrogenase derived from Lactobacillus and manufactured by TOYOBO Co., Ltd. was immobilized by glutaraldehyde solution by the same aforementioned procedure.

Response current value was measured at an interval of 15 seconds by preparing a reaction solution 5; adding it to the 0.084 unit-immobilized pyruvate dehydrogenase electrode; and carrying out the reaction for 3 minutes by applying +600 mV of impressed electric potential thereto using a silver (Ag)/silver chloride (AgCl) as the reference electrode and a Pt wire as the counter electrode. In this connection, the measurement was carried out under conditions of 25° C. and pH 7.0, and the measured ATP concentrations were 3 μM, 333 nM and 33 nM.

### <Reaction Solution 5>

(i) Magnesium chloride hexahydrate manufactured by Kanto Chemical Co., Inc.: 10 mM (final concentration)
(ii) Flavin adenine dinucleotide disodium salt manufactured by MP Biomedicals Inc.: 0.01 mM (final concentration)
(iii) Thiamin pyrophosphate chloride manufactured by MP Biomedicals Inc.: 0.2 mM (final concentration)
(iv) Adenosine 1-phosphate manufactured by Wako Pure Chemical Industries Ltd.: 0.33 mM (final concentration)
(v) Adenosine 3-phosphate manufactured by Wako Pure Chemical Industries Ltd.: 3.3 μM, 333 nM, 33 nM (final concentration)
(vi) Phosphoenolpyruvic acid manufactured by Wako Pure Chemical Industries Ltd.: 0.33 mM (final concentration)
(vii) Myokinase derived from chick muscle and manufactured by SIGMA ALDRICH Corporation: 0.29 U/ml (final concentration)
(viii) Pyruvate kinase derived from rubbit muscle and manufactured by MP Biomedicals Inc.: 5.2 U/ml (final concentration)
(ix) 1-Methoxy-5-methylphenaziniummethyl sulfate manufactured by Dojindo Laboratories: 10 mM (final concentration)
(x) Phosphate buffer pH 7.0; 50 mM (final concentration)

Response current was found in each sample from 1 minute after commencement of the reaction and it was able to obtain amplification curves according to the ATP concentrations in the same manner as in Example 1 (FIG. 5). Based on the above, it was revealed that ATP can also be detected when the pyruvate dehydrogenase is used in the Step C and an artificial electron mediator is used as the electron mediator. Additionally, since samples having different ATP concentrations show respectively different amplifications, it is shown that ATP can be measured quantitatively.

### (Measurement of ADP)

A pyruvate dehydrogenase electrode was prepared by immobilizing 0.096 unit of a pyruvate dehydrogenase derived from Lactobacillus and manufactured by TOYOBO Co., Ltd. using a glutaraldehyde solution by the same aforementioned procedure.

Response current value was measured at an interval of 30 seconds by preparing a reaction solution 6; adding it to the 0.096 unit-immobilized pyruvate dehydrogenase electrode; and carrying out the reaction for 4 minutes by applying +600 mV of impressed electric potential thereto using a silver (Ag)/silver chloride (AgCl) as the reference electrode and a Pt wire as the counter electrode. In this connection, the measurement was carried out under conditions of 25° C. and pH 7.0.

Also, although measured data on ATP has been shown in the previous Examples, 3 μM, 333 nM and 33 nM of ADP as one of the adenine nucleotides was measured this time.

### <Reaction Solution 6>

(i) Magnesium chloride hexahydrate manufactured by Kanto Chemical Co., Inc.: 10 mM (final concentration)
(ii) Flavin adenine dinucleotide disodium salt manufactured by MP Biomedicals Inc.: 0.01 mM (final concentration)
(iii) Thiamin pyrophosphate chloride manufactured by MP Biomedicals Inc.: 0.2 mM (final concentration)
(iv) Adenosine 1-phosphate manufactured by Wako Pure Chemical Industries Ltd.: 0.33 mM (final concentration)
(v) Adenosine 2-phosphate manufactured by Wako Pure Chemical Industries Ltd.: 3.3 μM, 333 nM, 33 nM (final concentration)
(vi) Phosphoenolpyruvic acid manufactured by Wako Pure Chemical Industries Ltd.: 0.33 mM (final concentration)
(vii) Myokinase derived from chick muscle and manufactured by SIGMA ALDRICH Corporation: 0.266 U/ml (final concentration)
(viii) Pyruvate kinase derived from rabbit muscle manufactured by MP Biomedicals Inc.: 3.37 U/ml (final concentration)
(ix) 1-Methoxy-5-methylphenaziniummethyl sulfate manufactured by Dojindo Laboratories: 10 mM (final concentration)
(x) Phosphate buffer pH 7.0; 50 mM (final concentration)

As a result, the response currents similar to the case of ATP measured in the previous Examples were confirmed in the samples of respective ADP concentrations and it was able to obtain amplification curves according to the respective ADP concentrations (FIG. 6). Based on the above, it can be said that a very small amount of adenine nucleotide can be electrochemically measured.

### EXAMPLE 3

In Example 1 and Example 2, it was shown that a very small amount of adenine nucleotide can be electrochemically measured by a system in which myokinase is used in the Step A, and pyruvate kinase in the Step B and pyruvate oxidase as a pyruvic acid oxidizing enzyme and pyruvate dehydrogenase in the Step C.

In Example 3, it is shown that adenine nucleotide can be measured by a system in which myokinase, hexokinase and glucose oxidase are respectively used as the enzymes to be used in the Steps A, B and C.

### <Enzyme Reaction Scheme 1>

Step A: ATP+AMP→2 ADP (enzyme E₁: myokinase)
Step B: ADP+D-glucose 6-phosphate→D-glucose+ATP (enzyme E₂: hexokinase)
Step C: D-glucose+O₂→D-glucono-1-lactone+H₂O₂ (enzyme E₃: glucose oxidase)

### (Preparation and Evaluation of Oxygen Electrode)

Glucose oxidases which are derived from yeast and equivalent to 3.71 units, 0.74 unit, 0.37 unit or 0.074 unit and manufactured by Oriental Yeast was added dropwise to the surface of a platinum (Pt) electrode having a diameter of 3 mm and air-dried overnight at 4° C. After the air-drying, glucose oxidase was immobilized to the electrode surface using a glutaraldehyde solution by the same procedure of Example 2.

Response current value at the time of the addition of glucose of each concentration was measured by applying +600 mV of impressed electric potential to the reaction solution 7 described in the following, using the glucose oxidase electrode, a silver (Ag)/silver chloride (AgCl) as the reference electrode and a Pt wire as the counter electrode. In this connection, the measurement was carried out under conditions of 25° C. and pH 7.0.

### <Reaction Solution 7>

(i) Magnesium chloride hexahydrate manufactured by Kanto Chemical Co., Inc.: 10 mM (final concentration)
(ii) Phosphate buffer pH 7.0: 50 mM (final concentration)

The results of carrying out the evaluation are shown in FIG. 7.

The electrodes to which respective concentrations of glucose oxidase was immobilized showed increase in the current value according to the glucose concentrations. In this connection, while detection limit of the electrode to which 3.71 unit of glucose oxidase was immobilized was 3 μM of glucose, detection limit of the electrode to which 0.74 unit or 0.37 unit of glucose oxidase was immobilized was 50 μM, and it was 500 μM regarding the 0.074 unit-immobilized electrode.

### (Measurement of ATP)

A glucose oxidase electrode was prepared by immobilizing 3.71 unit of a glucose oxidase derived from yeast and manufactured by Oriental Yeast Co., Ltd. using the glutaraldehyde solution by the same aforementioned procedure.

Response current value was measured at an interval of 30 seconds by preparing a reaction solution 8; adding it to the 3.71 unit glucose oxidase-immobilized enzyme electrode; and carrying out the reaction for 8 minutes by applying +600 mV of impressed electric potential thereto using a silver (Ag)/silver chloride (AgCl) as the reference electrode and a Pt wire as the counter electrode. In this connection, the measurement was carried out under conditions of 25° C. and pH 7.0, and 3 μM, 333 nM and 33 nM of ATP was measured.

### <Reaction Solution 8>

(i) Magnesium chloride hexahydrate manufactured by Kanto Chemical Co., Inc.: 10 mM (final concentration)
(ii) Adenosine 1-phosphate manufactured by Wako Pure Chemical Industries Ltd.: 0.33 mM (final concentration)
(iii) Adenosine 3-phosphate manufactured by Wako Pure Chemical Industries Ltd.: 3.3 μM, 333 nM, 33 nM (final concentration)
(iv) D-Glucose 6-phosphate monosodium salt manufactured by Wako Pure Chemical Industries Ltd.: 0.66 mM (final concentration)
(v) Myokinase derived from chick muscle and manufactured by SIGMA ALDRICH Corporation: 2.3 U/ml (final concentration)
(vi) Hexokinase derived from Yeast and manufactured by Oriental Yeast Co., Ltd.: 0.18 U/ml (final concentration)
(vii) Phosphate buffer pH 7.0: 50 mM (final concentration)

Response currents were found in the samples of respective ATP concentrations from 2 minutes after commencement of the reaction, and it was able to obtain amplification curves according to the respective ATP concentrations similar to the case of Examples 1 and 2 (FIG. 8). Based on the above, it is possible to measure ATP by a combination of enzymes, which is different from the ATP amplification reactions carried out in Examples 1 and 2 by a combination in which myokinase was used in the Step A, and pyruvate kinase in the Step B.

While the present invention has been described in detail and with reference to specific embodiments thereof, it will be apparent to one skilled in the art that various changes and modifications can be made therein without departing from the spirit and scope of the present invention.

This application is based on a Japanese patent application filed on Nov. 14, 2005, Japanese Patent Application No. 2005-328962, the entire contents thereof being thereby incorporated by reference.

### INDUSTRIAL APPLICABILITY

The features of the method for measuring adenine nucleotide of the present invention is that it can be applied to a high sensitivity electrochemistry type adenine nucleotide measuring apparatus which has a convenient and further miniaturized measuring device structure; is low in consumptive power; and does not require a treatment operation for substances which cause turbidity, and to an enzyme sensor. The method for measuring adenine nucleotide of the present invention is useful in such applications as ATP analysis in the field of food hygiene as an index of the degree of pollution with bacteria and the like microorganisms and of the food residues as a hotbed of microbial pollution, and as quality control of food including the degree of maturity, the degree of putrefaction and the like of food, water analysis for the water purification and the like.

1. A method for measuring adenine nucleotide, which comprises
a step A for converting adenosine triphosphate to adenosine diphosphate by an enzyme E₁;
a step B for converting said adenosine diphosphate and a phosphate donor P₂ to adenosine triphosphate and dephosphorylated phosphate donor P_{2′} by an enzyme E₂; and
a step C for electrochemically measuring said donor P_{2′} by carrying out an oxidation-reduction reaction of said donor P_{2′}.

2. The method for measuring adenine nucleotide according to claim 1, wherein the adenosine triphosphate and the dephosphorylated phosphate donor P_{2′} are formed according to the frequency of the reactions by repeating a cycle consisting of the step A and step B two or more times to carry out a reaction.

3. The method for measuring adenine nucleotide according to claim 1, wherein the dephosphorylated phosphate donor P_{2′} formed by step B is measured as the amount of adenine nucleotide.

4. The method for measuring adenine nucleotide according to claim 2, wherein myokinase is used as the enzyme E₁.

5. The method for measuring adenine nucleotide according to claim 1, wherein the oxidation-reduction reaction in the step C is carried out by an oxidation-reduction enzyme E₃.

6. The method for measuring adenine nucleotide according to claim 5, wherein the donor P_{2′} is electrochemically measured in the step C by carrying out an oxidation-reduction reaction of said donor P_{2′} which consumes oxygen molecule and detecting amount of the consumed oxygen.

7. The method for measuring adenine nucleotide according to claim 5, wherein the donor P_{2′} is electrochemically measured in the step C by carrying out an oxidation-reduction reaction of said donor P_{2′} which produces hydrogen peroxide and detecting amount of the produced hydrogen peroxide.

8. The method for measuring adenine nucleotide according to claim 5, wherein the electrochemically detecting way in the step C uses an electron mediator as the electron acceptor.

9. The method for measuring adenine nucleotide according to claim 1, wherein pyruvate kinase is used as the enzyme E₂, and pyruvate oxidase is used as the enzyme E₃.

10. The method for measuring adenine nucleotide according to claim 1, wherein pyruvate kinase is used as the enzyme E₂, and pyruvate dehydrogenase is used as the enzyme E₃.

11. The method for measuring adenine nucleotide according to claim 1, wherein hexokinase or glucokinase is used as the enzyme E₂, and glucose oxidase is used as the enzyme E₃.

12. The method for measuring adenine nucleotide according to claim 2, wherein the dephosphorylated phosphate donor P_{2′} formed by step B is measured as the amount of adenine nucleotide.

13. The method for measuring adenine nucleotide according to claim 3, wherein myokinase is used as the enzyme E₁.
